(19) United States
(12) Patent Application Publication
(10) Pub. No.: US 2007/0211213 A1
(43) Pub. Date: Sep. 13, 2007

(54) Ophthalmic instrument table and pads

(76) Inventor: Troy L. Carter, Abilene, TX (US)

Correspondence Address: STEVEN W. SMITH, 7237 BIRCHWOOD DRIVE, DALLAS, TX, 75240, US

(21) Appl. No.: 11/371,749

(22) Filed: Mar. 9, 2006

(51) Int. Cl. A61B 3/00

(52) U.S. Cl. 351/245

(57) ABSTRACT

An ophthalmic instrument table such as a slit-lamp table having pads attached to a top surface thereof. The pads provide a cushion for a doctor's elbows and/or forearms while the doctor utilizes the ophthalmic instrument to perform an ocular examination. Each pad includes a layer of padding material and an attachment mechanism for attaching the pad to the table. The pad may also include a firm substrate panel for supporting the layer of padding material, and a covering material for covering the padding material and the substrate panel. The pad may be shaped to conform to the side and front of the table, and to an ophthalmic instrument mounted on the table. The attachment mechanism enables a user to remove the pad from the table, use the pad on another table, or reposition the pad on the table by lifting the pad by hand.

### CROSS-REFERENCE TO RELATED APPLICATIONS

NOT APPLICABLE

### STATEMENT REGARDING FEDERALLY SPONSORED REASEARCH OR DEVELOPMENT

NOT APPLICABLE

### REFERENCE TO SEQUENCE LISTING, A TABLE, OR A COMPUTER PROGRAM LISTING COMPACT DISC APPENDIX

NOT APPLICABLE

### BACKGROUND OF THE INVENTION

The present invention relates to the field of ophthalmic instruments. More particularly, and not by way of limitation, the present invention is directed to an ophthalmic instrument table and pads for the top surface thereof. The pads are attached to the table in the areas where a doctor places his/her elbows or forearms while performing an ocular examination.

Diagnosis of eye disorders is initially based on the appearance of the eyes and on the symptoms that the person is experiencing. A variety of tests can then be carried out to confirm a problem or to determine the extent or severity of the disorder. For detailed visual examinations of the eye, the doctor may utilize an ophthalmic slit lamp. The slit lamp is a table-mounted binocular microscope that shines a light into the eye to allow the doctor to examine the entire eye under high magnification. The slit lamp has better optics than a handheld ophthalmoscope, providing magnification and a three-dimensional view, which allows measurement of depth. Often, eye drops are used to dilate the pupils so that the doctor can view even more of the eye, including the lens, vitreous humor, retina, and optic nerve.

FIG. 1 is an illustrative drawing of a conventional slit lamp 10 and slit-lamp table 11 being utilized to examine a patient's eye. The slit lamp is mounted on the top surface of a small table referred to as a slit-lamp table. During the examination, the patient places her chin on a chin support 12 and places her forehead against a headrest 13 so that her eye is kept stationary during the examination. A lamp 14 provides illumination, and a binocular microscope 15 provides magnification and a three-dimensional view of the interior of the patient's eye. The slit lamp includes various controls 16a and 16b that enable the doctor to adjust and control the slit lamp during the examination.

In order to properly adjust and control the slit lamp, the doctor must have a steady hand while focusing his attention on the image in the binocular microscope 15. This requires that the doctor either rest his elbows on the top surface of the slit-lamp table 11 or rest his forearms against the edge of the table. In either alternative, the hard top surface of the table, and the surface's sharp edges, quickly become painful irritants to the doctor. Slit-lamp tables are usually made of wood or composite materials covered by hard laminate. The edges of the top surface are squared and sharp. With time and repeated examinations, the hard surface of the table and its sharp edges cause pain and irritation to the doctor. The median nerves run along the undersurface of the forearms and are repeatedly exposed to pressure and repeated low levels of compression leading to fatigue and possibly to nerve damage.

What is needed in the art is an ophthalmic instrument table that is comfortable to use, and does not cause pain or irritation to the doctor during an ocular examination. Pads are also needed that are easily adjustable and removable so that they can be transferred to other types of ophthalmic instrument tables in the office of an eye care professional. The present invention provides such an ophthalmic instrument table and pads.

### BRIEF SUMMARY OF THE INVENTION

The present invention provides an ophthalmic instrument table configured with adjustable pads. In an exemplary embodiment described herein, the ophthalmic instrument table is a slit-lamp table. The pads provide a cushion for the doctor's elbows and/or forearms while the doctor utilizes the slit lamp to perform an ocular examination.

Thus, in one aspect, the present invention is directed to a pad for cushioning an ophthalmic instrument table. The pad includes a layer of padding material and an attachment mechanism for attaching the pad to a top surface of the table. In other embodiments, the pad may also include a firm substrate panel for supporting the layer of padding material, and a covering material for covering the padding material and the substrate panel. The pad may be shaped to conform to at least one side of the table, and to an ophthalmic instrument mounted on the table. The attachment mechanism may be constructed to enable a user to remove the pad from the ophthalmic instrument table or reposition the pad on the ophthalmic instrument table by lifting the pad by hand.

In another aspect, the present invention is directed to an ophthalmic instrument table having means for mounting an ophthalmic instrument on the top surface, and at least one pad attached to the top surface in a position suitable for cushioning a user's elbow or forearm while operating the ophthalmic instrument.

### BRIEF DESCRIPTION OF THE SEVERAL VIEWS OF THE DRAWING

In the following, the essential features of the invention will be described in detail by showing preferred embodiments, with reference to the figures of the attached drawings in which:

FIG. 1 (Prior Art) is an illustrative drawing of a conventional slit lamp and slit-lamp table being utilized to examine a patient's eye;

FIG. 2 is a perspective view of a slit-lamp table 21 in a first embodiment of the present invention;

FIG. 3 is a perspective view of the slit-lamp table of the present invention being utilized by a doctor;

FIG. 4 is another perspective view of the slit-lamp table of the present invention being utilized by a doctor;

FIG. 5 is another perspective view of the slit-lamp table of the present invention being utilized by a doctor;

FIG. 6 is a top view of a slit-lamp table pad 22 in one exemplary embodiment of the present invention;

FIG. 7 is a bottom view of the slit-lamp table pad 22 of FIG. 6; and

FIG. 8 is a cross-sectional view of the slit-lamp table pad 22 of FIG. 7 taken along the line 8-8.

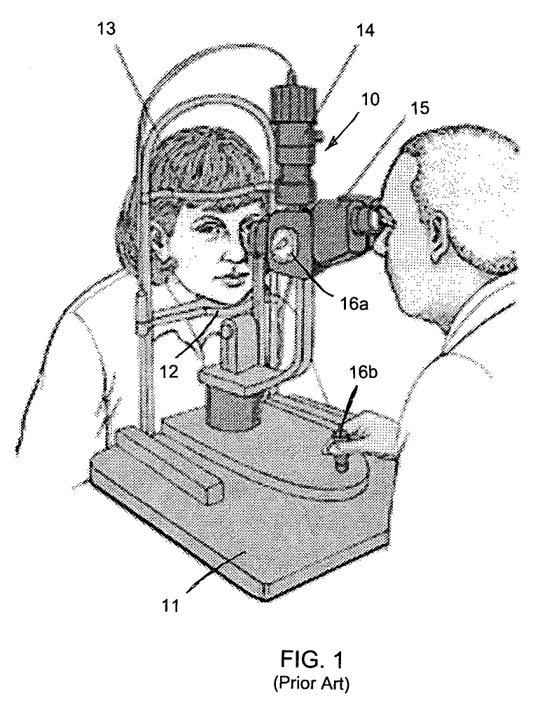

### DETAILED DESCRIPTION OF THE INVENTION

The present invention provides an ophthalmic instrument table configured with adjustable pads. In an exemplary embodiment described herein, the ophthalmic instrument table is a slit-lamp table. The pads provide a cushion for the doctor's elbows and/or forearms while the doctor utilizes the slit lamp to examine a patient's eye.

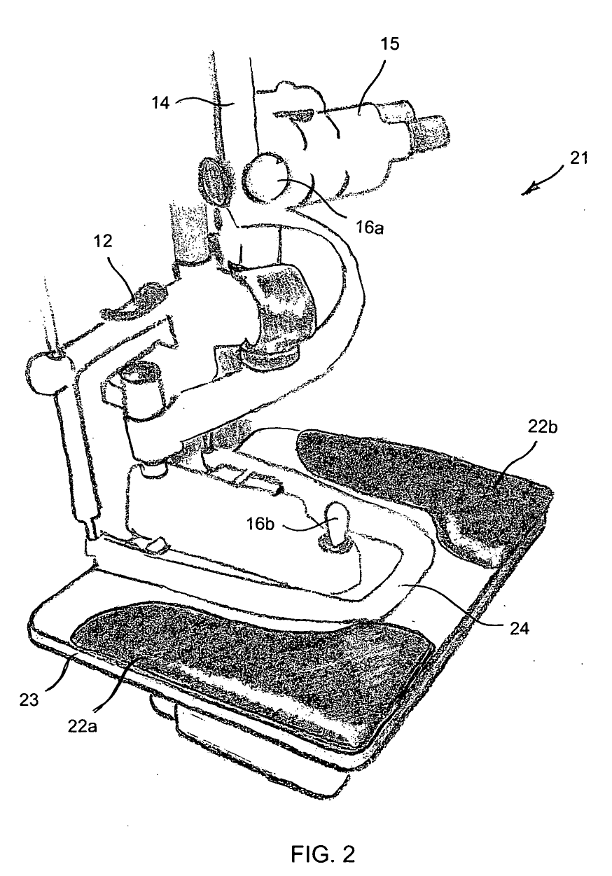

FIG. 2 is a perspective view of a slit-lamp table 21 in a first embodiment of the present invention. Two pads 22a and 22b are mounted on the top surface of the table. The pads are shaped to conform to the edge 23 of the table and to conform to the glide mechanism 24 of the slit lamp. The pads are also sized to cover all of the areas of the table surface where the doctor normally places his elbows or forearms during an ocular examination. In the preferred embodiment, the pads are positioned so that they extend slightly beyond the edges of the table, both on the sides and on the front of the table. In this way, the doctor's forearms are protected from the sharp edges of the top surface of the table.

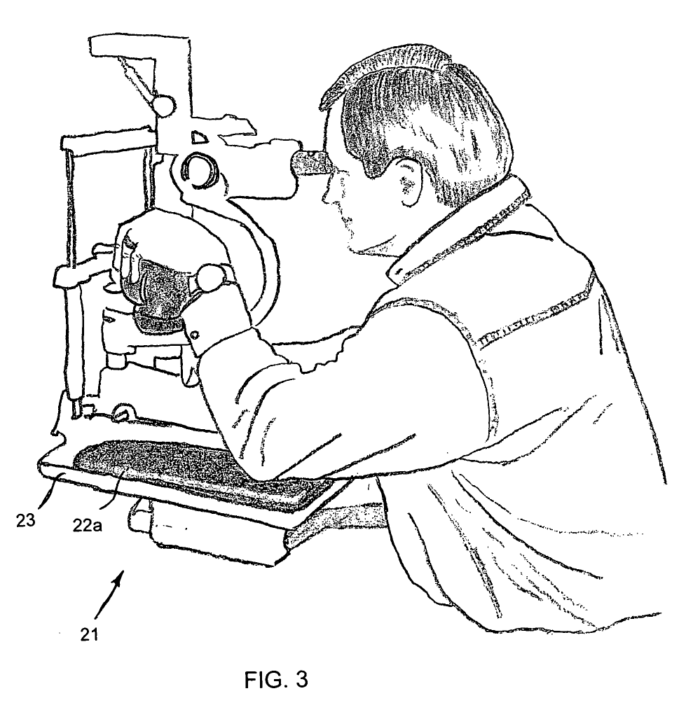

FIG. 3 is a perspective view of the slit-lamp table of the present invention being utilized by a doctor. The pad 22a is positioned so that the doctor can comfortably rest his elbow on the pad while adjusting and controlling the slit lamp.

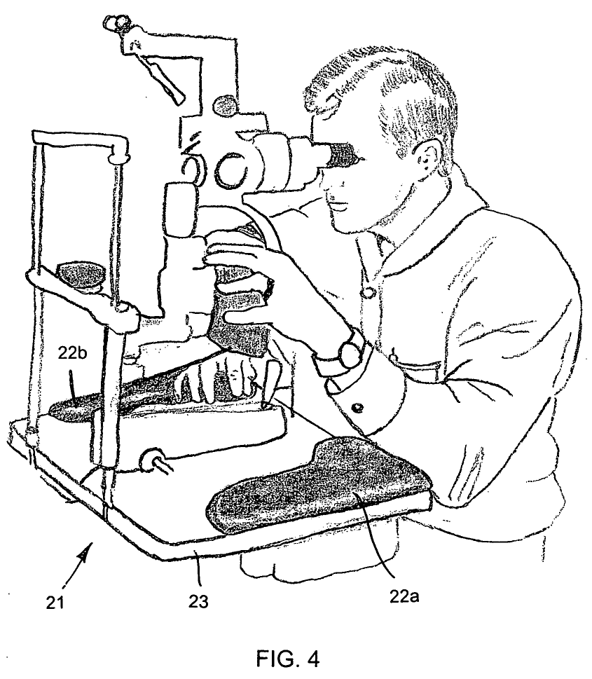

FIG. 4 is another perspective view of the slit-lamp table of the present invention being utilized by a doctor. In this view, the pads 22a and 22b are positioned so that they extend slightly beyond the front edge of the table. This feature enables the doctor to comfortably rest his forearms on the front of the pads while adjusting and controlling the slit lamp.

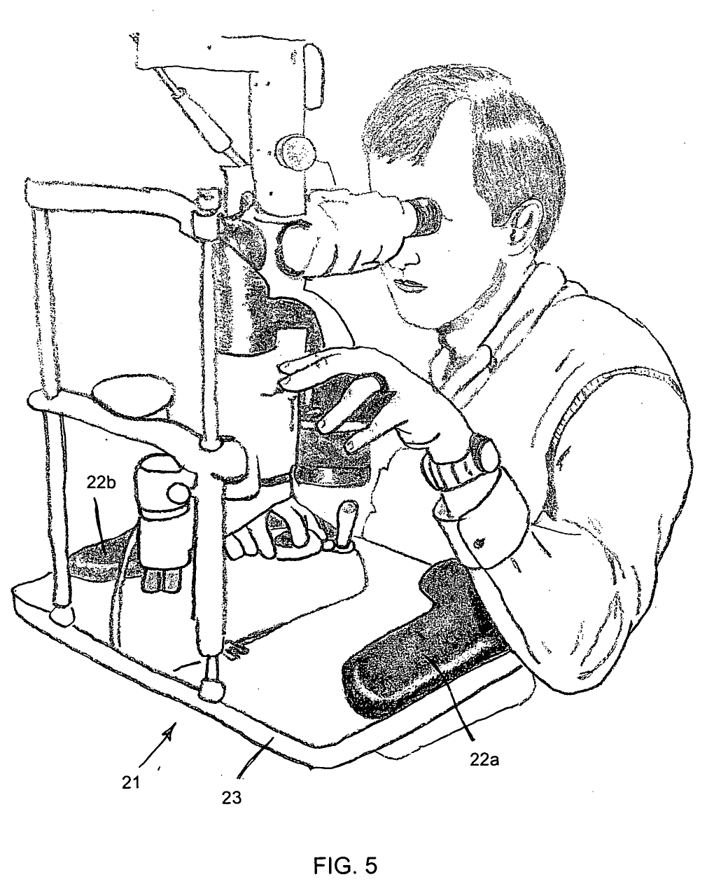

FIG. 5 is another perspective view of the slit-lamp table of the present invention being utilized by a doctor. In this view, the pads 22a and 22b are positioned so that they extend slightly beyond the side edges of the table. This feature enables the doctor to comfortably rest his forearms on the sides of the pads while adjusting and controlling the slit lamp. Thus, the pads relieve the repeated strain and discomfort of prior art slit-lamp tables, thereby allowing free and unimpeded maneuvering of the ophthalmic device.

There are a vast number of sizes and unique constructions of the various brands of ophthalmic slit lamps and slit-lamp tables. Because of this, it is necessary that the design of the slit-lamp pads be versatile. The basic design of the pads, therefore, has been developed to accommodate these variables. The pads also may be easily removed and reattached on the table surface in different areas or positions in order to facilitate the best examination and comfort of the doctor.

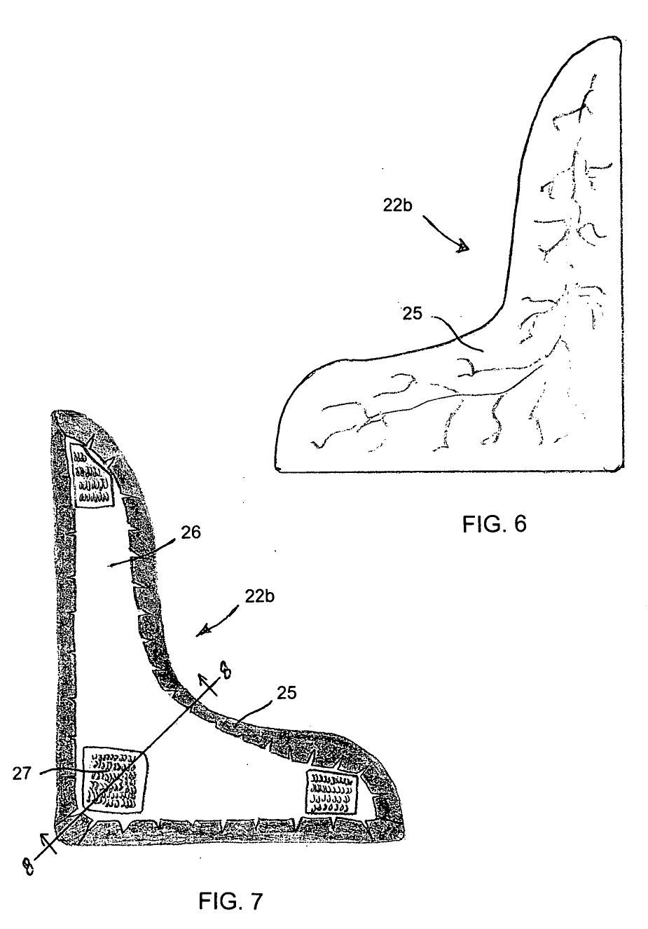

FIG. 6 is a top view of a slit-lamp table pad 22b in one exemplary embodiment of the present invention. In this embodiment, the pad is covered with a durable, yet comfortable, material 25 as described in more detail below.

FIG. 7 is a bottom view of the slit-lamp table pad 22b of FIG. 6. The bottom of a firm platform substrate 26 is visible, and the covering material 25 is attached thereto. A number of hook and loop patches 27 are also attached to the bottom of the substrate. The hook and loop patches perform in conjunction with complementary patches mounted on the surface of the slit-lamp table 21 to hold the pad in the desired position.

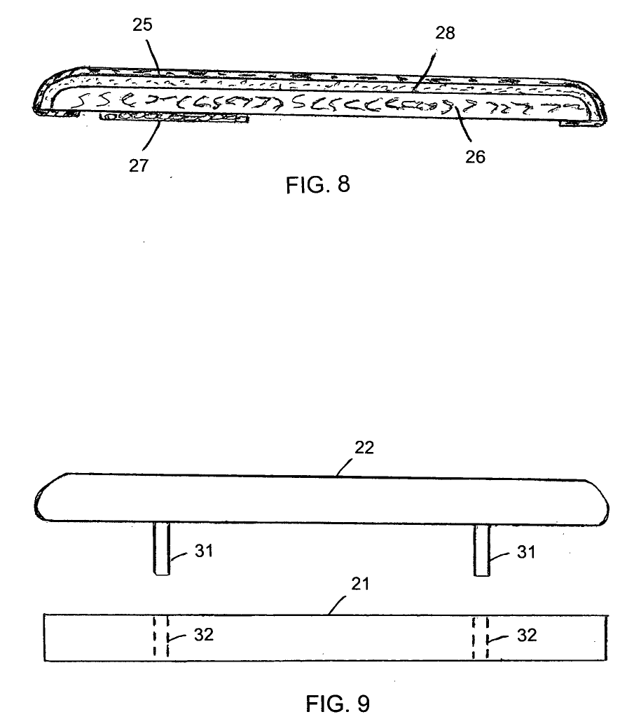

FIG. 8 is a cross-sectional view of the slit-lamp table pad 22 of FIG. 7 taken along the line 8-8. In this embodiment, the slit-lamp pad 22 has four components: the firm platform substrate 26, padding material 28, the covering material 25, and an attachment mechanism 27. All four components, however, may not be necessary for the creation of slit-lamp pads. For example, if the padding material is firm enough, then the platform substrate may be omitted without compromising the overall function of the pad. Additionally, if the surface of the padding material is sufficiently durable and has enough structural integrity to withstand repeated use, then the protective covering material may be omitted.

The platform substrate 26 may be constructed from a suitably firm material including, but not limited to plastic, wood, metal, rubber, synthetic laminates, cardboard, acrylic, vinyl, glass, fiberglass, foam, stone, clay, bone, cloth, various hard resins, plant fibers, and the like. The thickness of the substrate is preferably one inch or less. The shape of the substrate is uniquely designed to contour to the shape of the majority of slit-lamp tables, including a 90-degree corner or curve around the external edge of the substrate and a rounded or squared internal edge of the substrate contoured to avoid the glide mechanism of the slit lamp. The upper edge of the substrate may be rounded or smoothed although a squared edge may also be used. The shape of the substrate may alternatively be a square, rectangle, triangle, circle, oval, or an elongated strip along the anterior and lateral aspects of the slit-lamp table.

The padding material 28 is a layer placed on top of the substrate 26. The padding may be affixed to the substrate (for example with an adhesive) or may be held in place by the covering material 25. The padding provides the necessary cushioning effect to the slit-lamp pad. The padding is preferably the same size or slightly larger than the substrate and of the same general shape. The thickness of the padding is preferably in the range of 1/16 inch to one inch. The padding material may be, but is not limited to foam, rubber, acrylic, gel, animal feathers or fibers, plastic, plant fibers, leather, silicone, or a sealed bag with any of various types of liquid, water, or beads of any material.

The slit-lamp pads may be covered with a covering material 25 for support and protection of the underlying padding 28. The covering material may vary depending on the particular needs of the environment of the slit lamp. The covering material may be, but is not limited to vinyl, plastic, leather, naugahyde, acrylic, rubber, nylon, or fabric of various types. The color and texture of the material may vary depending on the preference of the consumer.

The slit-lamp pads are attached to the slit-lamp table 21 by any of various types of attachment mechanisms 27. The attachment mechanisms may include, but are not limited to hook and loop material, screws, bolts, hooks, glue, tape, clamps, nails, brackets, elastic straps, and the like. The hook and loop material is preferred because it is strong yet also provides an easy way of removal or repositioning on the table. This allows the slit-lamp pads to be repositioned in order to suit the needs of the anatomy of the doctor and to facilitate the best technique of examination.

In an alternative embodiment, each pad is equipped with at least two posts, which extend from the bottom surface of the pad. Holes are drilled through the top of the table in positions aligned with the posts. To attach the pads to the table, the posts are simply inserted into the holes. This method of attachment is sufficient because in use, the doctor's elbows or forearms press downward and/or sideways on the pads, but never upward. To remove the pads, the user simply lifts the pads by hand, thereby removing the posts from the holes. In this embodiment, the pads may be easily removed and transferred to another ophthalmic instrument table having similarly drilled holes. However, the ability to make fine adjustments in the position of the pads is lost.

The slit-lamp pads 22 are constructed by first choosing a suitable platform substrate 26. For example, a suitable substrate may be a ⅛-inch thick red oak panel. A template measuring the exact size of the desired slit-lamp pad may be used to lay out the desired shape of the pad on the flat surface of the wood panel. The substrate is then cut from the wood panel. The upper edge may be sanded and rounded. The use of other substrate materials may require different cutting devices or large multiple cutting templates to pressure cut the substrates in multiples.

The padding 28 may also be cut in a like manner depending on the material. Ideally, the padding should be slightly larger than the substrate 26 so that the padding may be rounded over the edges of the substrate for comfort. The padding may then be glued or taped onto the substrate. Once the substrate is padded, the protective covering material 25 may be smoothly wrapped over it and glued or stapled or tacked to the bottom of the substrate.

The attachment mechanism 27 is then formed. All forms of attachment mechanisms described above may be utilized to suit the needs of particular table designs. As noted, the hook and loop material is preferred because it enables the pads to be easily removed or repositioned. The hook and loop material is also easy to apply to both the bottom of the substrate 26 and the top surface of the table 21. Industrial strength hook and loop material is readily available with the back surface pre-treated with an adhesive. The user simply peels off a protective covering to expose the adhesive, and applies a patch of the hook and loop material to the surface of the table in a position aligning with the complementary patch on the bottom of the pad. The slit-lamp pad is then positioned and pushed onto the slit-lamp table in the area desired for its use. To reposition or remove the pad, the user need only pull up on an edge of the pad to separate the hook and loop patches.

It should be noted that although the preferred embodiment has been described in the context of a slit-lamp table, the pads described herein may also be used with a variety of other tables. For example, an eye care professional has many other applications for removable pads including use on keratometer tables, ophthalmic camera tables, auto-refractor tables, lensometer tables, ophthalmic laser tables, corneal topography tables, nerve fiber analyzer tables, orb scan tables, air tonometry tables, and the like. Most of these, and other ophthalmic device tables, have a similar design and overall shape. Therefore, they would also benefit from the use of removable pads. Thus, the pads of the present invention may be mounted on any or all of these other tables in the office of an eye care professional. Alternatively, a user may simply remove the pads from one table and attach them to a pre-positioned hook and loop patch on another device table instead of attaching pads on all device tables in a particular office setting.

Although preferred embodiments of the present invention have been illustrated in the accompanying drawings and described in the foregoing Detailed Description, it is understood that the invention is not limited to the embodiments disclosed, but is capable of numerous rearrangements, modifications, and substitutions without departing from the scope of the invention. The specification contemplates any all modifications that fall within the scope of the invention defined by the following claims.

1. A pad for cushioning an ophthalmic instrument table, said pad comprising:
a layer of padding material covering a portion of a top surface of the table extending substantially from a front edge of the table to a base of an ophthalmic instrument mounted on said table, wherein the padding material covers the portion of the top surface of the table where a user places his elbows while operating the ophthalmic instrument; and
an attachment mechanism for attaching the padding material in a stationary position on the top surface of the table.

2. The pad according to claim 1, wherein the pad is shaped to conform to the front and sides of the table.

3. The pad according to claim 1, wherein the pad is shaped to conform to the ophthalmic instrument mounted on the table.

4. The pad according to claim 1, wherein the attachment mechanism enables a user to remove the pad from the ophthalmic instrument table or reposition the pad on the ophthalmic instrument table by lifting the pad by hand.

5-6. (canceled)

7. The pad according to claim 1, further comprising:
a firm substrate panel for supporting the layer of padding material; and
a covering material for covering the padding material and the substrate panel.

8. The pad according to claim 7, wherein the layer of padding material is attached to the substrate panel with an adhesive.

9. The pad according to claim 7, wherein the top edges of the substrate panel are rounded.

10. The pad according to claim 7, wherein the layer of padding material is constructed of a material selected from a group consisting of:
foam, rubber, acrylic, gel, animal feathers or fibers, plastic, plant fibers, leather, silicone, a sealed bag filled with a liquid, and a sealed bag filled with beads.

11. The pad according to claim 7, wherein the substrate panel is constructed of a firm material selected from a group consisting of:
plastic, wood, metal, rubber, synthetic laminates, cardboard, acrylic, vinyl, glass, fiberglass, foam, stone, clay, bone, cloth, a hard resin, and plant fibers.

12. The pad according to claim 7, wherein the covering material is constructed of a material selected from a group consisting of:
vinyl, plastic, leather, naugahyde, acrylic, rubber, nylon, and fabric.

13. An ophthalmic instrument table having a top surface, said table comprising:
means for mounting an ophthalmic instrument on the top surface; and
at least one pad attached to the top surface in a stationary position suitable for cushioning a user's elbow or forearm while operating the ophthalmic instrument.

14. The ophthalmic instrument table according to claim 13, wherein the pad extends substantially from at least one side of the table to a base of the ophthalmic instrument mounted on the table, wherein the pad is shaped to conform to the at least one side of the table and to conform to the base of the ophthalmic instrument mounted on the table.

15. The ophthalmic instrument table according to claim 13, wherein the pad includes an attachment mechanism for attaching the pad to the top surface of the table, wherein the attachment mechanism enables a user to remove the pad from the ophthalmic instrument table or reposition the pad on the top surface of the ophthalmic instrument table by lifting the pad by hand.

16. The ophthalmic instrument table according to claim 15, wherein the attachment mechanism includes:
a first piece of hook and loop material attached to a bottom surface of the pad; and
a second, complementary piece of hook and loop material attached to the top surface of the table.

17. The ophthalmic instrument table according to claim 15, wherein at least two apertures are drilled in the top surface of the ophthalmic instrument table in the position of the pad, and the attachment mechanism includes two posts extending from a bottom surface of the pad, wherein the two posts are positioned to align with the two apertures in the top surface of the ophthalmic instrument table.

18. The ophthalmic instrument table according to claim 15, wherein the pad also includes:
a firm substrate panel;
a layer of padding material on top of the substrate panel; and
a covering material for covering the padding material and the substrate panel.

19. The ophthalmic instrument table according to claim 18, wherein the layer of padding material is attached to the substrate panel with an adhesive.

20. The ophthalmic instrument table according to claim 18, wherein the covering material is attached to the substrate panel, wherein the covering material holds the padding material on top of the substrate panel by encasing the padding material and attaching to the substrate panel.
